(19) United States
(12) Patent Application Publication
(10) Pub. No.: US 2025/0361281 A1
(43) Pub. Date: Nov. 27, 2025

(54) AMYLIN RECEPTOR (hAMY3R) AGONISTS WITH IMPROVED CHEMICAL STABILITY

(71) Applicant: GUBRA A/S, Hørsholm (DK)

(72) Inventors: Anthony MURRAY, Hørsholm (DK); Kristoffer Tobias Gustav RIGBOLT, Hørsholm (DK); Paola MAGOTTI, Vanløse (DK); Søren Ljungberg PEDERSEN, BROUP (DK); Morten LUNDH, Hørsholm (DK); Borja BALLARÍN-GONZÁLEZ, Hørsholm (DK); Jens Christian Frøslev NIELSEN, Hørsholm (DK)

(73) Assignee: GUBRA A/S, Hørsholm (DK)

(21) Appl. No.: 18/873,302

(22) PCT Filed: Jun. 30, 2023
(86) PCT No.: PCT/EP2023/068022

§ 371 Date: Dec. 9, 2024

(30) Foreign Application Priority Data
Jul. 1, 2022 (EP) 22182529.2

(51) Int. Cl. C07K 14/575; A61K 38/00

(52) U.S. Cl. CPC C07K 14/575; A61K 38/00

(57) ABSTRACT

The present invention relates to polypeptides acting as amylin receptor (hAMY3R) agonists with improved chemical stability.

### FIELD OF THE INVENTION

The present invention relates to polypeptides based on the sequence of the human adrenomedullin fragment hAM15-52, acting as amylin receptor (hAMY3R) agonists. In particular, the present invention relates to polypeptides with improved chemical stability.

### BACKGROUND OF THE INVENTION

Obesity is a medical condition in which excess body fat has accumulated to the extent that it has a negative impact on health. It is affecting a huge number of individuals worldwide and increasing rapidly in certain parts of the world. The World Health Organisation (WHO) estimated that in 2016, approximately 650 million people were obese worldwide. Obesity is defined as a body mass index (BMI) above 30. Obesity is considered a major risk factor for developing a variety of medical conditions, such as cardiovascular diseases (e.g. hypertension, atherosclerosis, heart attacks or stroke), NASH, musculoskeletal disorders, certain kinds of cancers, depression, and diabetes type II, and hence is detrimental to human health. Cardiovascular diseases and diabetes are two main diseases associated with obesity. A large amount of research has been conducted in the obesity field in search for new treatments for obesity or obesity-related diseases and disorders.

Diabetes is a group of metabolic disorders characterized by a high blood sugar level. As of 2019, the International Diabetes Federation estimated that 463 million people are suffering from diabetes worldwide, approximately half of the individuals being diagnosed. Diabetes is divided into two types, namely type I and type II diabetes. Type I diabetes results from the pancreas's failure to produce enough insulin due to loss of beta cells caused by an autoimmune response. On the other hand, type II diabetes is a condition that begins with insulin resistance in which cells fail to respond to insulin properly and as the disease progresses may also result in a lack of insulin.

### the Calcitonin Peptide Family

The calcitonin family of peptides consists of the hormone peptides calcitonin (CT), calcitonin gene-related peptide (CGRP), islet amyloid polypeptide (IAPP, amylin or hAMY₁₋₃₇), and adrenomedullin (hAM) as well as their precursors. hAMY₁₋₃₇ is a 37-residue peptide hormone that is co-secreted with insulin from the pancreatic β-cells with the amino acid sequence Lys-Cys-Asn-Thr-Ala-Thr-Cys-Ala-Thr-Gln-Arg-Leu-Ala-Asn-Phe-Leu-Val-His-Ser-Ser-Asn-Asn-Phe-Gly-Ala-Ile-Leu-Ser-Ser-Thr-Asn-Val-Gly-Ser-Asn-Thr-Tyr. Amylin suppresses glucagon release and inhibits gastric emptying and hence plays an important role in maintaining glucose homeostasis by decreasing the blood sugar concentration. Furthermore, amylin has shown to reduce food intake and plays an important role in satiety, making it a potential candidate for treating e.g. obesity and/or diabetes. hAM is a 52-residues peptide hormone expressed in all tissues with the amino acid sequence Tyr-Arg-Gln-Ser-Met-Asn-Asn-Phe-Gln-Gly-Leu-Arg-Ser-Phe-Gly-Cys-Arg-Phe-Gly-Thr-Cys-Thr-Val-Gln-Lys-Leu-Ala-His-Gln-Ile-Tyr-Gln-Phe-Thr-Asp-Lys-Asp-Lys-Asp-Asn-Val-Ala-Pro-Arg-Ser-Lys-Ile-Ser-Pro-Gln-Gly-Tyr. It is a potent vasodilator and has shown positive influence in cardiovascular diseases, such as myocardial infarction, limb ischemia, and hypertension.

The biological activity of the calcitonin protein family is generally mediated via binding to the calcitonin receptor (CTR) and the calcitonin receptor, like receptor (CRLR), both belonging to family 2 of the G-protein-coupled receptors (GPCR). These receptors may be co-expressed in combination with different receptor modifying proteins (RAMP1-3) generating functional receptors for the individual peptides in the calcitonin protein family. Co-expression of CTR with RAMP1 leads to the formation of a receptor for amylin and CGRP (AMY1R), co-expression of CTR with RAMP2 leads to the amylin receptor 2 (AMY2R), and co-expression of CTR with RAMP3 leads to the amylin receptor (AMY3R). Co-expression of CRLR with RAMP1 leads to a formation of a receptor for CGRP (CGRP1R), co-expression of CRLR with RAMP2 leads to a formation of a receptor for adrenomedullin (AM1R) and co-expression of CRLR with RAMP3 leads to a formation of a receptor for adrenomedullin and CGRP (AM2R).

Several of the native peptides in the calcitonin protein family show considerable overlap in pharmacology between receptors. For example, adrenomedullin is approximately 100 times less potent on AMY3R compared to hAMY₁₋₃₇. The adrenomedullin fragment (hAM15-52) is almost equipotent on AMY3R and AM1R with an EC₅₀ of 1.3 nM on AMY3R and an EC₅₀ value of 1.1 nM on AM1R (said EC₅₀ value being measured according to the examples herein). hAMY₁₋₃₇, on the other hand, has an EC₅₀ value of 10 pM on AMY3R while being inactive on AM1R.

Human amylin (hAMY₁₋₃₇ or amylin) possesses some drawbacks, such as a high tendency of fibrillation, a short in vivo half-life, and chemical instability at pH 7. Thus, native amylin is suboptimal for use as a pharmaceutically active ingredient. Some of the drawbacks of native amylin have been overcome by the successful amylin analogue Pramlintide, which has been approved by the FDA for use in type I and type II diabetes. However, Pramlintide is formulated at pH 4, as it fibrillates at pH 7, which may cause pain at the injection site upon administration. Contrary to hAMY₁₋₃₇, the human adrenomedullin fragment hAM15-52 does not fibrillate at pH 7. Therefore, the hAM15-52 backbone has previously been utilized in the development of new amylin analogues (see WO 2022/063925 A1). It has shown that a few specific substitutions in hAM15-52 can convert hAM15-52 into a selective amylin receptor agonist by completely abolishing its adrenomedullin receptor (AM1R) potency while simultaneously enhancing its amylin receptor (hAMY3R) potency. The advantage of this chemical strategy is that such amylin receptor agonists resemble hAMY₁₋₃₇ in terms of pharmacodynamics but benefit from the low tendency of fibrillation inherent to hAM15-52. One such example is the polypeptide with the amino acid sequence KCNTATCTVQRLAEQIAQFTDKDKDNVAPPTNVGSNGHyp (SEQ ID NO: 3) having a hAMY3R EC₅₀ of 14 nM and an hAM1R EC₅₀ of >5000 nM. Albeit its high in vitro potency for hAMY3R, its low fibrillation tendency, and its high in vivo efficacy, this peptide was found to be susceptible to chemical instability due to deamidation, dimerization and isomerization. The present invention addresses these drawbacks of SEQ ID NO: 3 in order to provide a polypeptide with improved chemical stability that is optimal for clinical development.

### SUMMARY OF THE INVENTION

The present invention relates to the finding that the chemical stability of SEQ ID NO: 3 can be improved, without adversely affecting other properties such as potency, by substituting the asparagine (N) in position X₃, X₃₂ and X₃₆ of SEQ ID NO: 3 with a leucine (L), an alanine (A) and an alanine (A) respectively, thereby avoiding deamidation. The present invention further relates to the finding that the chemical stability of SEQ ID NO: 3 can be improved, without adversely affecting other properties such as potency, by replacing the disulfide bridge (—S—S—) of SEQ ID NO: 3 with a methylene bridge (—S—CH₂—S—), thereby suppressing dimerization and the formation of high molecular weight products. The present invention further relates to the finding that the chemical stability can be improved, without adversely affecting other properties such as potency, by substituting the aspartic acid (D) in position 25 of SEQ ID NO: 3 with glutamic acid (E), thereby avoiding isomerization.

Thus, in a first aspect the present invention relates to a polypeptide or a pharmaceutically acceptable salt thereof comprising the amino acid sequence KCLTATCTVARLAEQIAQFTDKDKENVAPPTAVGSAG[Hyp] (SEQ ID NO: 4), or a derivative thereof having up to 2 amino acid substitutions with the proviso that the substitutions are not present in any of the positions X₂-X₄, X₇, X₁₁, X₃₂ or X₃₆₋₃₈ of SEQ ID NO:4.

### Definitions

In the present context, the polypeptides are generally amidated at the C-terminal (—CONH₂), like the native peptides; amylin and adrenomedullin. However, the polypeptides of the present invention may also have either a free carboxylic acid (—COOH) or another post-translational modification, such as a methyl ester (—COOMe). In a highly preferred embodiment of the invention, the polypeptides are amidated at the C-terminal. The polypeptides according to the present invention may have a free amine (—NH₂), be N-acylated (—NHCOR), N-methylated (—NHCH₃ or —N(CH₃)₂), deaminated at the N-terminal, or N-lipidated.

In the present context, lipidation refers to the covalent attachment of a lipid to a polypeptide, such as C18DA (octadecanedioic acid), C20DA (icosanedioic acid) optionally through linker/spacer consisting of one or more covalently connected units commonly used such as [^{γ}E], [OEG] or [AHX] as illustrated below.

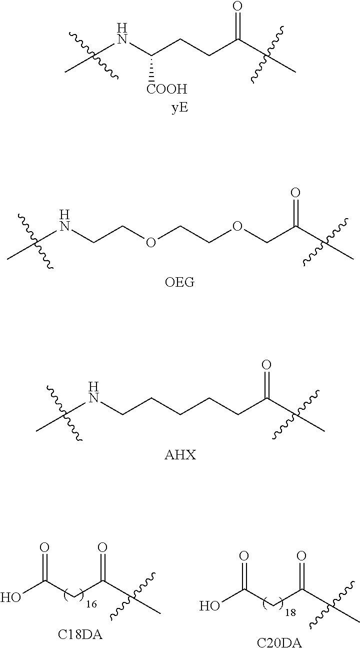

Lipidation is typically performed to improve the pharmacokinetic profile of a polypeptide by e.g. improving metabolic stability, reducing enzymatic degradation, lowering excretion and metabolism, all in all resulting in a prolonged in vivo half-life (t_{1/2}). The polypeptides according to the invention may be lipidated or non-lipidated depending on the desired half-life. The polypeptides may be lipidated, e.g. at a lysine (K) residue or at the N-terminal as exemplified herein. Preferably, the lipid (and linker) is selected from the list consisting of tetradecanoic acid (C14), hexadecanoic acid (C16), C18DA[^{γ}E]-, C18DA[^{γ}E][^{γ}E]-, C18DA[^{γ}E][OEG]-, C18DA[^{γ}E][OEG][OEG]-, C18DA[^{γ}E][^{γ}E][OEG][OEG]-, C18DA[^{γ}E][AHX]-, C18DA[^{γ}E][^{γ}E][AHX]-, C20DA[^{γ}E]-, C20DA[^{γ}E][^{γ}E]-, C20DA[^{γ}E][OEG]-, C20DA[^{γ}E][OEG][OEG]-, C20DA[^{γ}E][^{γ}E][OEG][OEG]-, C20DA[^{γ}E][AHX]-, or C20DA[^{γ}E][^{γ}E][AHX]-. Most preferably, the lipidation is C20DA[^{γ}E]-. Most preferably, the lipidation is performed at the N-terminal.

In the present context, EC₅₀ values are used as a measure of agonist potency at a given receptor. An EC₅₀ value is a measure of the concentration of a compound required to achieve half of that compound's maximal activity in a particular assay.

In the present context, a polypeptide or derivative thereof may be in the form of a pharmaceutically acceptable salt. Thus, pharmaceutically acceptable salts are intended to include any salts that are commonly used in formulations of peptides. Such salts include both acid addition salts and basic salts, and examples may be found e.g. in Remington's Pharmaceutical Sciences, 17^{th} edition. Likewise, various solvates of the hAM15-52 analogues or pharmaceutically acceptable salts thereof are also within the scope of the invention.

In the present context, unless otherwise stated, the amino acids are all L-amino acids (L-stereoisomer, natural amino acids). The abbreviation Hyp refers to L-hydroxyproline. Substitutions in a derivative may be substitutions to natural amino acids as well as unnatural amino acids, including L- and D-stereoisomers. Preferably, a substitution in a variant is a conservative substitution to a conservative amino acid. The groups of conservative amino acids may be defined as:
G, A, V, L, I, P (aliphatic or cyclic),
S, C, T, M (hydroxyl or sulphur containing)
F, Y, W (aromatic)
H, K, R (basic)
D, E, N, Q (acidic or amide)

In the present context, it should be understood that the cysteines in position X₂ and X₇ are covalently connected by a bridge, such as a disulfide bridge (—S—S—) or methylene bridge (—S—CH₂—S—). Most preferably, the cysteines in position X₂ and X₇ are covalently connected by a methylene bridge (—S—CH₂—S—).

In the present context, the term “treatment” should be understood in the broadest sense as prevention, amelioration, or treatment. Thus, treatment is also intended to include prophylactic treatment.

### DETAILED DESCRIPTION OF THE INVENTION

### Aspect 1—Polypeptides

The polypeptide with the amino acid sequence KCNTATCTVQRLAEQIAQFTDKDKDNVAPPTNVGSNGHyp (SEQ ID NO: 3) has previously shown to possess high in vitro potency for hAMY3R (i.e. hAMY3R EC₅₀ of 0.014 nM), high selectivity towards hAMY3R over hAM1R (i.e. hAM1R EC₅₀ of >5000 nM), and no fibrillation. Furthermore, the current inventors found that SEQ ID NO: 3 also possesses high in vivo efficacy. Albeit its positive properties, the present invention arises from the finding that SEQ ID NO: 3 is prone to chemical instability, which poses a major concern in terms of long-term stability of a drug candidate. The present invention relates to ways of improving the chemical stability of SEQ ID NO: 3 and structurally related polypeptides.

The present inventors found that the chemical instability of SEQ ID NO: 3 was caused by deamidation, dimerization, and isomerization. SEQ ID NO: 3 contains seven potential deamidation sites (i.e. N in position X₃, X₂₆, X₃₂ and X₃₆; Q in position X₁₀, X₁₅, X₁₈), three of which (i.e. position X₃, X₃₂ and X₃₆) were found to be hot-spots for deamidation, as illustrated below in scheme 1. Thus, the present inventors found that the chemical stability of SEQ ID NO: 3 could be greatly improved, without adversely affecting the potency (see example 1, Table 1), by substituting the asparagines (N) in position 3, 32 and 36 of SEQ ID NO: 3 with leucine (L), and alanine (A) as shown in scheme 1, in order to prevent deamidation.

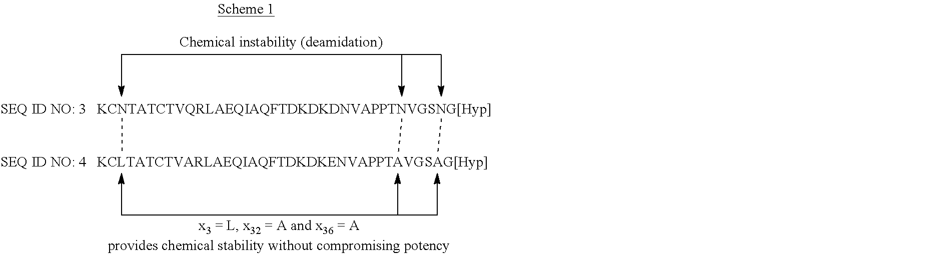

The present inventors further found that a further cause of the chemical instability of SEQ ID NO: 3 could be attributed to isomerization. In particular, the inventors found that the aspartic acid (D) in position X₂₅ of SEQ ID NO: 3 was slightly prone to structural and chiral isomerization, as illustrated in scheme 2 below, which also reduced the yield in the synthesis.

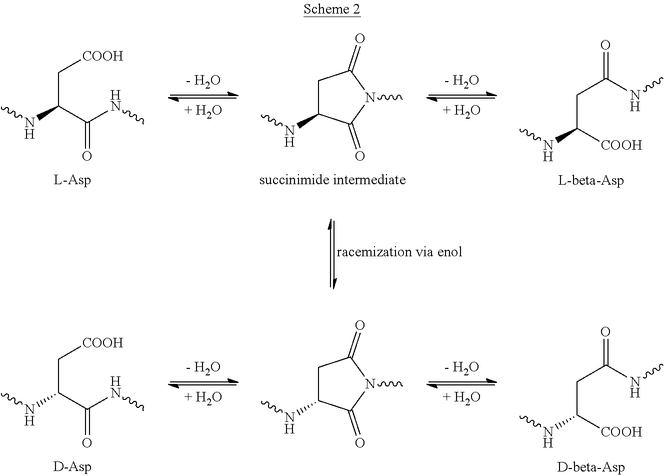

Thus, the present invention further relates to the finding that isomerization could be prevented and that the overall synthesis yield increased by substitution of aspartic acid (D) in position X₂₅ of SEQ ID NO: 3 with a glutamic acid (E) as illustrated in Scheme 3 below, without adversely affecting the potency (see example 1, Table 1).

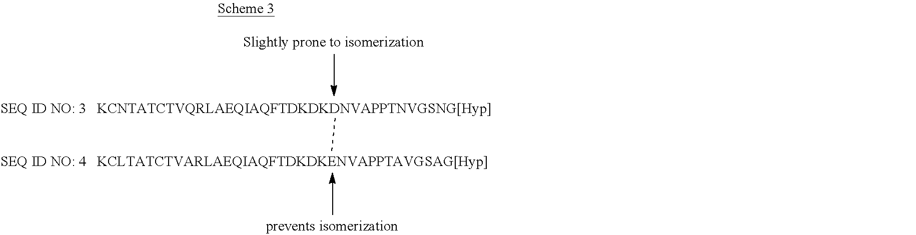

Thus, in a first aspect the present invention relates to a polypeptide or a pharmaceutically acceptable salt thereof comprising the amino acid sequence:
KCLTATCTVARLAEQIAQFTDKDKENVAPPTAVGSAG[Hyp] (SEQ ID NO: 4) or a derivative thereof having up to 2 amino acid substitutions with the proviso that the substitution(s) is/are not present in any of the positions X₂-X₄, X₇, X₁₁, X₃₂ or X₃₆₋₃₈.

Preferably, a derivative of SEQ ID NO:4 has 1 amino acid substitution. Most preferably, there is no substitution(s) present in SEQ ID NO: 4.

Thus, in a preferred embodiment, the present invention relates to a polypeptide or a pharmaceutically acceptable salt thereof comprising the amino acid sequence:
KCLTATCTVARLAEQIAQFTDKDKENVAPPTAVGSAG[Hyp] (SEQ ID NO: 4) or a derivative thereof having 1 amino acid substitution with the proviso that the substitution is not present in any of the positions X₂-X₄, X₇, X₁₁, X₃₂ or X₃₆-38.

In a more preferred embodiment, the present invention relates to a polypeptide or a pharmaceutically acceptable salt thereof comprising the amino acid sequence:
KCLTATCTVARLAEQIAQFTDKDKENVAPPTAVGSAG[Hyp].

The present inventors further found that the chemical instability of SEQ ID NO: 3 could to some degree be attributed to dimerization, as illustrated in scheme 3 below. Thus, the present invention further relates to the finding that dimerization can be suppressed by replacing the disulfide bridge (—S—S—), formed between the cysteines in position X₂ and X₇ of SEQ ID NO: 3, with a methylene bridge (—S—CH₂—S—), to improve the chemical stability (see example 1, Table 1).

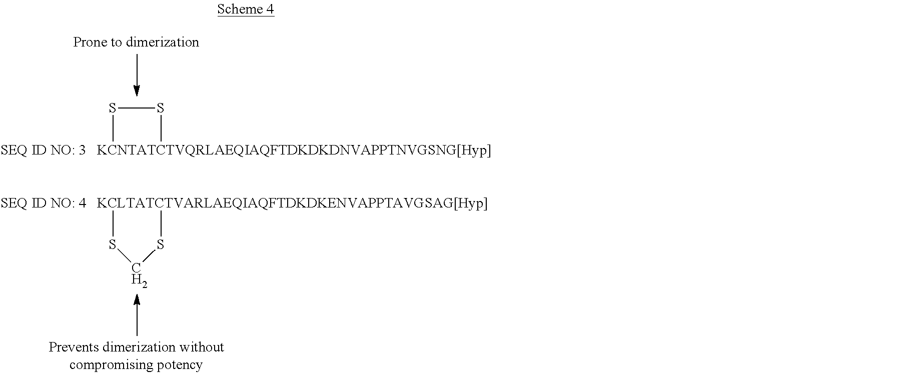

Thus, in an embodiment of the invention, the cysteine in X₂ and X₇ are covalently connected through a disulfide bridge (—S—S—). In the most preferred embodiment of the invention, the cysteine in X₂ and X₇ are covalently connected through a methylene bridge (—S—CH₂—S—) in order to minimize dimerization and prevent formation of high molecular weight products.

Thus, in a more preferred embodiment, the present invention relates to a polypeptide or a pharmaceutically acceptable salt thereof comprising the amino acid sequence:
KCLTATCTVARLAEQIAQFTDKDKENVAPPTAVGSAG[Hyp] (SEQ ID NO: 4) or a derivative thereof having up to 2 amino acid substitutions with the proviso that the substitutions are not present in any of the positions X₂-X₄, X₇, X₁₁, X₃₂ or X₃₆-38, and wherein the cysteines in X₂ and X₇ are covalently connected through a methylene bridge (—S—CH₂—S—).

In an even more preferred embodiment, the present invention relates to a polypeptide or a pharmaceutically acceptable salt thereof comprising the amino acid sequence:
KCLTATCTVARLAEQIAQFTDKDKENVAPPTAVGSAG[Hyp] (SEQ ID NO: 4) or a derivative thereof having 1 amino acid substitution with the proviso that the substitution is not present in any of the positions X₂-X₄, X₇, X₁₁, X₃₂ or X₃₆₋₃₈, and wherein the cysteines in X₂ and X₇ are covalently connected through a methylene bridge (—S—CH₂—S—).

In an even more preferred embodiment, the present invention relates to a polypeptide or a pharmaceutically acceptable salt thereof comprising the amino acid sequence:
KCLTATCTVARLAEQIAQFTDKDKENVAPPTAVGSAG[Hyp] (SEQ ID NO: 4), wherein the cysteines in X₂ and X₇ are covalently connected through a methylene bridge (—S—CH₂—S—).

The polypeptides according to the invention may be lipidated with various lipids depending on the desired half-life of the polypeptides. Preferably, the lipid is selected from the list consisting of tetradecanoic acid (C14)-, hexadecanoic acid (C16)-, C18DA[^{γ}E]-, C18DA[^{γ}E][^{γ}E]-, C18DA[^{γ}E][OEG]-, C18DA[^{γ}E][OEG][OEG]-, C18DA[^{γ}E][^{γ}E][OEG][OEG]-, C18DA[^{γ}E][AHX]-, C18DA[^{γ}E][^{γ}E][AHX]-, C20DA[^{γ}E]-, C20DA[^{γ}E][^{γ}E]-, C20DA[^{γ}E][OEG]-, C20DA[^{γ}E][OEG][OEG]-, C20DA[^{γ}E][^{γ}E][OEG][OEG]-, C20DA[^{γ}E][AHX]-, or C20DA[^{γ}E][^{γ}E][AHX]-. Most preferably, the lipid is C20DA[^{γ}E]-. The polypeptides may be lipidated at e.g. a lysine residue (K) or at the N-terminal. Most preferably, the polypeptides are lipidated at the N-terminal. In a most preferred embodiment, the N-terminal is lipidated with C20DA[^{γ}E]-.

Thus, in an even more preferred embodiment, the present invention relates to a polypeptide or a pharmaceutically acceptable salt thereof comprising the amino acid sequence:
KCLTATCTVARLAEQIAQFTDKDKENVAPPTAVGSAG[Hyp] (SEQ ID NO: 4) or a derivative thereof having up to 2 amino acid substitutions with the proviso that the substitutions are not present in any of the positions X₂-X₄, X₇, X₁₁, X₃₂ or X₃₆-38, wherein the cysteines in X₂ and X₇ are covalently connected through a disulfide bridge (—S—S—) or a methylene bridge (—S—CH₂—S—), most preferably a methylene bridge (—S—CH₂—S—) and further wherein the polypeptide is lipidated with a lipid selected from the list consisting of tetradecanoic acid (C14)-, hexadecanoic acid (C16)-, C18DA[^{γ}E]-, C18DA[^{γ}E][^{γ}E]-, C18DA[^{γ}E][OEG]-, C18DA[^{γ}E][OEG][OEG]-, C18DA[^{γ}E][^{γ}E][OEG][OEG]-, C18DA[^{γ}E][AHX]-, C18DA[^{γ}E][^{γ}E][AHX]-, C20DA[^{γ}E]-, C20DA[^{γ}E][^{γ}E]-, C20DA[^{γ}E][OEG]-, C20DA[^{γ}E][OEG][OEG]-, C20DA[^{γ}E][^{γ}E][OEG][OEG]-, C20DA[^{γ}E][AHX]-, or C20DA[^{γ}E][^{γ}E][AHX]-, most preferably C20DA[^{γ}E]-.

In yet an even more preferred embodiment, the present invention relates to a polypeptide or a pharmaceutically acceptable salt thereof comprising the amino acid sequence:
KCLTATCTVARLAEQIAQFTDKDKENVAPPTAVGSAG[Hyp] (SEQ ID NO: 4) or a derivative thereof having 1 amino acid substitutions with the proviso that the substitutions are not present in any of the positions X₂-X₄, X₇, X₁₁, X₃₂ or X₃₆-38, and wherein the cysteines in X₂ and X₇ are covalently connected through a disulfide bridge (—S—S—) or a methylene bridge (—S—CH₂—S—), most preferably a methylene bridge (—S—CH₂—S—), and further wherein the polypeptide is lipidated with a lipid selected from the list consisting of tetradecanoic acid (C14)-, hexadecanoic acid (C16)-, C18DA[^{γ}E]-, C18DA[^{γ}E][^{γ}E]-, C18DA[^{γ}E][OEG]-, C18DA[^{γ}E][OEG][OEG]-, C18DA[^{γ}E][^{γ}E][OEG][OEG]-, C18DA[^{γ}E][AHX]-, C18DA[^{γ}E][^{γ}E][AHX]-, C20DA[^{γ}E]-, C20DA[^{γ}E][^{γ}E]-, C20DA[^{γ}E][OEG]-, C20DA[^{γ}E][OEG][OEG]-, C20DA[^{γ}E][^{γ}E][OEG][OEG]-, C20DA[^{γ}E][AHX]-, or C20DA[^{γ}E][^{γ}E][AHX]-, most preferably C20DA[^{γ}E]-.

In a highly preferred embodiment, the present invention relates to a polypeptide or a pharmaceutically acceptable salt thereof comprising the amino acid sequence:
KCLTATCTVARLAEQIAQFTDKDKENVAPPTAVGSAG[Hyp], wherein the cysteines in X₂ and X₇ are covalently connected through a disulfide bridge (—S—S—) or a methylene bridge (—S—CH₂—S—), most preferably a methylene bridge (—S—CH₂—S—), and further wherein the polypeptide is lipidated with a lipid selected from the list consisting of tetradecanoic acid (C14)-, hexadecanoic acid (C16)-, C18DA[^{γ}E]-, C18DA[^{γ}E][^{γ}E]-, C18DA[^{γ}E][OEG]-, C18DA[^{γ}E][OEG][OEG]-, C18DA[^{γ}E][^{γ}E][OEG][OEG]-, C18DA[^{γ}E][AHX]-, C18DA[^{γ}E][^{γ}E][AHX]-, C20DA[^{γ}E]-, C20DA[^{γ}E][^{γ}E]-, C20DA[^{γ}E][OEG]-, C20DA[^{γ}E][OEG][OEG]-, C20DA[^{γ}E][^{γ}E][OEG][OEG]-, C20DA[^{γ}E][AHX]-, or C20DA[^{γ}E][^{γ}E][AHX]-, most preferably C20DA[^{γ}E]-.

In any of the above embodiments, wherein the polypeptides are lipidated, most preferably the lipidation is an N-terminal lipidation and most preferably, the cysteines in X₂ and X₇ are covalently connected through a methylene bridge (—S—CH₂—S—).

In a more highly preferred embodiment, the present invention relates to a polypeptide or a pharmaceutically acceptable salt thereof comprising the structure:

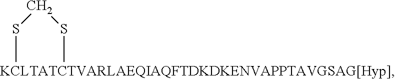

wherein the polypeptide is lipidated with a lipid selected from the list consisting of tetradecanoic acid (C14)-, hexadecanoic acid (C16)-, C18DA[^{γ}E]-, C18DA[^{γ}E][^{γ}E]-, C18DA[^{γ}E][OEG]-, C18DA[^{γ}E][OEG][OEG]-, C18DA[^{γ}E][^{γ}E][OEG][OEG]-, C18DA[^{γ}E][AHX]-, C18DA[^{γ}E][^{γ}E][AHX]-, C20DA[^{γ}E]-, C20DA[^{γ}E][^{γ}E]-, C20DA[^{γ}E][OEG]-, C20DA[^{γ}E][OEG][OEG]-, C20DA[^{γ}E][^{γ}E][OEG][OEG]-, C20DA[^{γ}E][AHX]-, or C20DA[^{γ}E][^{γ}E][AHX]-, most preferably C20DA[^{γ}E]-.

In an even more highly preferred embodiment, the present invention relates to a polypeptide or a pharmaceutically acceptable salt thereof comprising the structure,

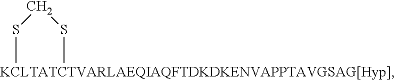

wherein the polypeptide is lipidated at the N-terminal with a lipid selected from the list consisting of tetradecanoic acid (C14)-, hexadecanoic acid (C16)-, C18DA[^{γ}E]-, C18DA[^{γ}E][^{γ}E]-, C18DA[^{γ}E][OEG]-, C18DA[^{γ}E][OEG][OEG]-, C18DA[^{γ}E][^{γ}E][OEG][OEG]-, C18DA[^{γ}E][AHX]-, C18DA[^{γ}E][^{γ}E][AHX]-, C20DA[^{γ}E]-, C20DA[^{γ}E][^{γ}E]-, C20DA[^{γ}E][OEG]-, C20DA[^{γ}E][OEG][OEG]-, C20DA[^{γ}E][^{γ}E][OEG][OEG]-, C20DA[^{γ}E][AHX]-, or C20DA[^{γ}E][^{γ}E][AHX]-, most preferably C20DA[^{γ}E]-.

In any of the above embodiments, preferably the C-terminal is amidated (—CONH₂).

In a most preferred embodiment, the present invention relates to a polypeptide with the structure,

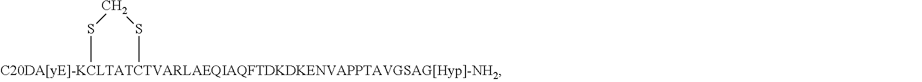

or a pharmaceutically acceptable salt thereof.

### Aspect II—Medical Use

In a second aspect, the invention relates to a polypeptide according to the first aspect for use as a medicament. More particularly, the second aspect of the invention relates to a polypeptide according to the first aspect for use in treating, preventing, or ameliorating a disease, disorder, or condition selected from the list consisting of excess food intake, excess body weight, obesity, Binge eating disorder, Prader-Willi syndrome, dyslipidemia, metabolic diseases/disorders, diabetes I or II, impaired glucose tolerance, insulin resistance syndrome and/or NASH, preferably obesity, diabetes, NASH or combinations thereof, most preferably obesity and/or diabetes.

### Aspect III—Pharmaceutical Compositions

In a third aspect, the present invention relates to a pharmaceutical composition comprising one or more polypeptide(s) according to the first aspect and/or its medical use(s) in treating, preventing, or ameliorating a disease, disorder, or condition according to the second aspect. A pharmaceutical composition may comprise a pharmaceutically acceptable carrier (vehicle) and/or one or more excipient(s) in accordance with conventional techniques in the art, such as those disclosed in ‘Remington: Essentials of Pharmaceutics’, Ed. by Linda A. Felton, Pharmaceutical press 2012. Suitable formulations include but are not limited to tablets, pills, capsules, emulsions, suspensions, sustained release, solutions, or freeze-dried powder intended for dissolution prior to administration. It should be appreciated that different routes of administration may be used depending on the choice of formulation. Such administration routes may include but are not limited to oral administration, parenteral administration (intravenous (IV), subcutaneous (SC), intradermal (ID) and intramuscular (IM)), or inhalation. Preferably, the administration route is parental administration. Most preferably, the administration route is subcutaneous.

### Aspect IV—Method of Treatment

In a fourth aspect, the invention relates to a method of treating a human or animal subject with one or more polypeptide(s) according to the first aspect or a pharmaceutical composition according to the third aspect, wherein the human or animal subject is diagnosed with, suffering from, or at risk of developing one or more of the diseases, disorders, or conditions according to the second aspect. The method involves administering one or more compounds according to a first aspect or a pharmaceutical composition according to the third aspect in an effective therapeutic amount to treat diseases, disorders or conditions mentioned in the third aspect.

### EXAMPLES

### General Protocols for Synthesis of the Hybrid Polypeptides

The peptides were synthesized using a Syroll fully automated parallel peptide synthesizer (MultiSynTech GmbH, Germany) equipped with heating block, on Tentagel S RAM with a loading of 0.23-0.25 mmol/g (Rapp polymer GmbH, Germany). Na-Fmoc deprotection was performed in two stages by treating the resin with 40% piperidine/DMF (0.2 M HOBt (1-hydroxybenzotriazole)) for 3 min at 45° C. followed by 20% piperidine/DMF (0.1 M HOBt) for 7-12 min at 75° C. Except Asp, Cys and His residues which were Na-Fmoc deprotections at room temperature; i.e. 40% piperidine/DMF (0.2 M HOBt) for 3 min followed by 20% piperidine/DMF (0.1 M HOBt) for 15 min. The coupling chemistry was DIC (N,N′-diisopropyl-carbodiimide)/Oxyma (ethyl cyano(hydroxyimino)acetate) in DMF using amino acid solutions of 0.5 M in DMF and a molar excess of 6-fold. Standard Fmoc protected amino acids were used. Coupling conditions was single or double couplings for 15 min at 75° C. Except His and Cys residues, which were double coupled for 15 min at 50° C. The Fmoc-amino acids were dissolved at 0.5 M in DMF containing 0.5 M Oxyma, except His which was dissolved in NMP. The resin was washed 4× with NMP after Na-Fmoc deprotection and 3× after couplings.

The disulfide bridge was formed on the resin by using Trityl (Trt) as the protecting group for cysteine and treating with 1% iodine in 75% HFIP (Hexafluoroisopropanol) in DCM for 1 min. The resin was washed 3× with 75% HFIP in DCM followed by 4×DCM.

After synthesis, the resin was washed with DCM and dried, and the polypeptide was cleaved from the resin by a 35 min treatment with TFA (trifluoroacetic acid)/TES (triethylsilane)/water (95/2.5/2.5) at 42° C., followed by precipitation with 4 volumes of cold diethyl ether, further washing with diethyl ether and left to dry.

The methylene bridge between the two cysteines was prepared in solution after cleavage of the peptide from resin using standard procedures. The peptide (300 mg) was dissolved in 20 mM phosphate buffer (10 mL, pH 7.8), and dithiothreitol (2 equiv) was added and the mixture stirred for 10 min. Sodium iodide (2 equiv) was then added, followed by a solution of diiodomethane (40 equiv) and ethanolamine (20 equivalents) in acetonitrile (15 mL). The reaction was monitored by LCMS until complete, diluted with 100 ml water and lipophilic material was removed by extraction with diethyl ether (2×80 mL)

Crude peptides were purified by reverse phase HPLC using a Waters preparative HPLC with C8 column (Reprosil Gold 200 Å, 5 μm, 40 mm×250 mm), preparative pumps (waters 2545), UV/VIS detector (Waters 2489) and a Waters fraction collector III. The mobile phase was run with a gradient of buffer A (0.1% TFA in H₂O) and buffer B (0.1% TFA in ACN, gradient: 35-45% B over 20 min) at a flow rate of 50 ml/min at RT. Relevant fractions were analysed, pooled, and lyophilized. The final product was characterized by analytical UPLC-MS.

Peptide purity and mass were determined by analytical HPLC-MS on a Kinetex C8 column (Phenomenex, 100 Å, 2.6 μm, 4.6 mm×150 mm) using a Waters Acquity HPLC System equipped with 3100 Mass Detector. Analysis was performed by gradient elution with buffer A (0.3% TFA in H₂O) and buffer B (0.3% TFA in ACN) at a temperature of 40° C. Details of the gradient is summarized below.

### Waters HPLC Method (21 Min Run) Acetonitrile Gradient:

10% to 40% (0-2 min), 40% to 60% (2 min to 16 min), 60% to 90% (16.5 min-18.5 min), 90% to 10% (18.5-21 min).

### Example 1

### SEQ ID 4:

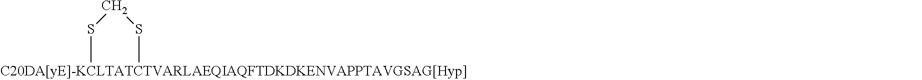

Retention Time 10.9 min

Found m/2=2193.2, m/3=1462.6, m/4=1096.9. Calc m=4384

General Protocols for cAMP Assays for Measuring In Vitro Receptor Activation
hAMY3-R:

Cells stably overexpressing the hAMY3 receptor were obtained from Ogeda (now Astellas Pharma), sub-cloned and a monoclonal cell-line with an appropriate assay-window was expanded, aliquoted and frozen. An aliquot was thawed and plated in DPBS with 0.05% casein and 0.5 mM IBMX as 2000 cells/well in a 384-well format. The cells were then immediately stimulated for 30 min at room temperature with graded doses of test compound using human amylin (Bachem, cat no. H-7905) as a positive control. cAMP accumulation was measured using a Cisbio assay for Gs coupled receptors (cat. no. 62AM4PEC), where the assay reagents were added as per the manufacturer's instructions and time-resolved fluorescence energy transfer recorded after one hour.

hAM1-R:

Cells stably overexpressing the hAM1 receptor were obtained from Ogeda (now Astellas Pharma), sub-cloned and a monoclonal cell-line with an appropriate assay-window was expanded, aliquoted and frozen. An aliquot was thawed and plated in DPBS with 0.05% casein and 0.5 mM IBMX as 8000 cells/well in a 384-well format. The cells were then immediately stimulated for 30 min at room temperature with graded doses of test compound using human adrenomedullin 1-52 (Bachem, cat no. H-2932) as a positive control. cAMP accumulation was measured using a Cisbio assay for Gs coupled receptors (cat. no. 62AM4PEC), where the assay reagents were added as per the manufacturer's instructions and time-resolved fluorescence energy transfer recorded after one hour.

### General Protocols for Determination of Physical Stability of Peptide Analogues

Peptides were dissolved in buffers (50 mM sodium acetate at pH 4 or 50 mM sodium phosphate at pH 7) and incubated for one hour. The samples were then divided into two replicates of 80 μl in a black 384 well plate (μ-clear, Greiner Bio-One) and mixed with Thioflavin T (ThT) to a final concentration of 4 μM. The plate was sealed and placed in a plate reader (CLARIOstar, BMG) where the temperature was set at 40° C. during the course of the experiment. The samples were further stressed by shaking the plate at 700 rpm (linear) for five minutes before every measurement. The fluorescence was measured every 10 min for four days by exciting the ThT at 450 nm and measuring the emission at 480 nm. For each peptide, the ThT signal over time was smoothened using Local Polynomial Regression fitting (LOESS) as implemented in the statistical programming environment R. For the smoothened data, the maximum ThT signal was normalized in percent between the maximum ThT signal for hAMY and the buffer background. Thus, high values indicate fibrillation properties similar to hAMY while low values indicate no fibrillation.

### General Protocol for Determination of Deamidation and Isomerization

Peptides were dissolved in 50 mM phosphate buffer (pH 7) and incubated at 40° C. for up to 28 days. The samples were then analysed on a Exploris120 mass spectrometer coupled to a Vanquish Tandem UPLC (both Thermo Fisher) equipped with an Acquity BEH C18 column (1.7 um, 2.1×50 mm, 130A, Waters). Peptides were eluted using a linear gradient starting at 5% over 7.5 min to 70% buffer B at a flow rate of 0.4 mL/min (buffer B=100% acetonitrile with 0.1% formic acid; buffer A=100% water with 0.1% formic acid). Data were acquired in data-dependent acquisition mode at a MS1 resolution of 60.000, and a MS2 resolution of 30.000 in positive mode (Top4). Peptides were fragmented with a normalized collision energy of 25%. Deamidation and isomerization were quantified using area under the curve integration.

### General Protocol for Determination of Dimer and HMWP Formation

Peptides were dissolved in 50 mM phosphate buffer (pH 7) and incubated at 40° C. for up to 28 days. The samples were then analysed using size exclusion chromatography analysis to quantify the amount of High Molecular Weight Products (HMWP), which is an overall term for covalently linked peptide dimers, trimers, and higher order multimers. Analyses were carried out on a Dionex Ultimate 3000 RS UHPLC Focused chromatography system (Thermo Scientific) equipped with a Acquity Protein BEH SEC 1.7 μm 4.6×300 mm 125 Å column (Waters part no 186006506). The solvent system consisted of two mobile phases that are assumed to break all non-covalent interactions between peptides leaving only monomeric peptide and covalent HMWP to be analysed.
Mobile phase A (all v/v %): 95% H2O, 5% MeCN (Acetonitrile), 0.1% TFA (Trifluoroacetic acid)
Mobile phase B (all v/v %): 95% MeCN, 5% H2O, 0.1% TFA

The typically load was 1 μl of 1.0 mg/ml, and the detection signal at 215 nm should optimally be between 0.1 and 1.5 abs units. Isocratic elution was done with 60% mobile phase B and a flow of 0.3 ml/min in 20 minutes. Chromatograms were integrated as HMWP (all peaks in front of the main peak) The amount of the various species was reported as area relative to total area in percentage.

TABLE 1

| | | | | | | Dimerization | |
|---|---|---|---|---|---|---|---|
| | hAMY3R | hAM1R | | Fibril | | and HMWP | |
| | EC₅₀ | EC₅₀ | N-terminal | formation | Deamidation | formation | Isomerization |
| SEQ ID NO: | (nM) | (nM) | Lipidation | (%) | (%) | (%) | (%) |
| 1 | 1.274 | 1.113 | None | No | — | — | — |
| (hAM₁₅₋₅₂) | | | | | | | |
| 2 | 0.01 | >5000 | None | Yes | — | — | — |
| (hAMY₁₋₃₇) | | | | | | | |
| 3 | 0.014 | >5000 | C20DA[yGlu]− | No | 97 | 12 | 5 |
| 4 | 0.009 | >5000 | C20DA[yGlu]− | No | <5 | 2 | 1.2 |

Table 1 shows in vitro potency, fibrillation, (%) deamidation, (%) dimerization and HMWP formation, and (%) isomerization. (%) deamidation for SEQ ID NO: 3 is calculated based on the sum of polypeptides wherein deamidation occurred in either position X₃ or X₃₆ or in both X₃ and X₃₆. From SEQ ID NO: 4 having less than <5% deamidation position X₁₅, X₁₈, and X₂₆ is not particular prone to deamidation.

TABLE 2

| SEQ ID NO: | 1 | 2 | 3 | 4 | 5 | 6 | 7 | 8 | 9 | 10 | 11 | 12 | 13 | 14 | 15 | 16 | 17 | 18 | 19 | 20 |
|---|---|---|---|---|---|---|---|---|---|---|---|---|---|---|---|---|---|---|---|---|
| 1 | G | C | R | F | G | T | C | T | V | Q | K | L | A | H | Q | I | Y | Q | F | T |
| (hAM₁₅₋₅₂) | | | | | | | | | | | | | | | | | | | | |
| 2 | K | C | N | T | A | T | C | A | T | Q | R | L | A | N | F | L | V | H | S | S |
| (hAMY₁₋₃₇) | | | | | | | | | | | | | | | | | | | | |
| 3 | K | C | N | T | A | T | C | T | V | Q | R | L | A | E | Q | I | A | Q | F | T |
| 4 | K | C | L | T | A | T | C | T | V | A | R | L | A | E | Q | I | A | Q | F | T |

| SEQ ID NO: | 21 | 22 | 23 | 24 | 25 | 26 | 27 | 28 | 29 | 30 | 31 | 32 | 33 | 34 | 35 | 36 | 37 | 38 |
|---|---|---|---|---|---|---|---|---|---|---|---|---|---|---|---|---|---|---|
| 1 | D | K | D | K | D | N | V | A | P | R | S | K | I | S | P | Q | G | Y |
| (hAM₁₅₋₅₂) | | | | | | | | | | | | | | | | | | |
| 2 | N | N | F | — | G | A | I | L | S | S | T | N | V | G | S | N | T | Y |
| (hAMY₁₋₃₇) | | | | | | | | | | | | | | | | | | |
| 3 | D | K | D | K | D | N | V | A | P | P | T | N | V | G | S | N | G | Hyp |
| 4 | D | K | D | K | E | N | V | A | P | P | T | A | V | G | S | A | G | Hyp |

Table 2 shows the sequences of hAM15-52, hAMY₁₋₃₇, the reference peptide from prior art (SEQ ID NO: 3) and a polypeptide according to the invention (SEQ ID NO: 4).

1-11. (canceled)

12. A polypeptide or a pharmaceutically acceptable salt thereof comprising the amino acid sequence KCLTATCTVARLAEQIAQFTDKDKENVAPPTAVGSAG[Hyp], or a derivative thereof having up to 2 amino acid substitutions with the proviso that the substitutions are not present in any of the positions X₂-X₄, X₇, X₁₁, X₃₂ or X₃₆₋₃₈.

13. A polypeptide according to claim 12, wherein the derivative has 1 amino acid substitution.

14. A polypeptide according to claim 12, wherein the cysteine in X₂ and X₇ are covalently connected through a disulfide bridge (—S—S—) or methylene bridge (—S—CH₂—S—).

15. A polypeptide according to claim 12, wherein the cysteine in X₂ and X₇ are covalently connected through a methylene bridge (—S—CH₂—S—).

16. A polypeptide according to claim 12, wherein the polypeptide is lipidated with a lipid selected from the list consisting of tetradecanoic acid (C14)-, hexadecanoic acid (C16)-, C18DA[^{γ}E]-, C18DA[^{γ}E][^{γ}E]-, C18DA[^{γ}E][OEG]-, C18DA[^{γ}E][OEG][OEG]-, C18DA[^{γ}E][^{γ}E][OEG][OEG]-, C18DA[^{γ}E][AHX]-, C18DA[^{γ}E][^{γ}E][AHX]-, C20DA[^{γ}E]-, C20DA[^{γ}E][^{γ}E]-, C20DA[^{γ}E][OEG]-, C20DA[^{γ}E][OEG][OEG]-, C20DA[^{γ}E][^{γ}E][OEG][OEG]-, C20DA[^{γ}E][AHX]-, or C20DA[^{γ}E][^{γ}E][AHX]-.

17. A polypeptide according to claim 16, wherein the lipid is C20DA[^{γ}E]-.

18. A polypeptide according to claim 12, wherein the polypeptide is N-terminal lipidated.

19. A polypeptide according to claim 12 having the structure, 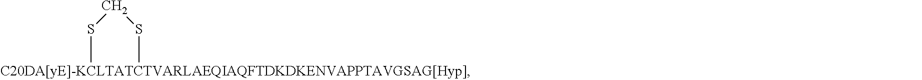
or a pharmaceutically acceptable salt thereof.

20. A polypeptide according to claim 12, wherein the C-terminal is amidated (—CONH₂).

21. A polypeptide according to claim 12, for use as a medicament.

22. A pharmaceutical composition comprising a polypeptide or a pharmaceutically acceptable salt thereof according to claim 12, a pharmaceutically acceptable carrier, and optionally one or more excipients.


SEQUENCE LISTING (5 sequences)

SEQ ID NO: 1 - protein, 38 aa, synthetic construct
GCRFGTCTVQKLAHQIYQFTDKDKDNVAPRSKISPQGY

SEQ ID NO: 2 - protein, 37 aa, Homo sapiens [sequence omitted]

SEQ ID NO: 3 - protein, 38 aa, synthetic construct
  SITE 38: note = L-hydroxyproline
  DISULFID 2..7: note = disulfide bridge (-S-S-)
  LIPID 1: note = C20DA[gammaGlu]- (attached to the nitrogen at the N-terminal)
KCNTATCTVQRLAEQIAQFTDKDKDNVAPPTNVGSNGX

SEQ ID NO: 4 - protein, 38 aa, synthetic construct
  SITE 38: note = L-hydroxyproline
  DISULFID 2..7: note = methylene bridge (-S-CH2-S-)
  LIPID 1: note = C20DA[gammaGlu]- (attached to the nitrogen at the N-terminal)
KCLTATCTVARLAEQIAQFTDKDKENVAPPTAVGSAGX

SEQ ID NO: 5 - protein, 52 aa, Homo sapiens
YRQSMNNFQGLRSFGCRFGTCTVQKLAHQIYQFTDKDKDNVAPRSKISPQGY